(19) United States
(12) Patent Application Publication
(10) Pub. No.: US 2026/0167914 A1
(43) Pub. Date: Jun. 18, 2026

(54) Magnetically Sealed Organ on Chip Platform for Rapid Disassembly

(71) Applicant: Northeastern University, Boston, MA (US)

(72) Inventors: Ryan A. Koppes, Charlestown, MA (US); Abigail N. Koppes, Charlestown, MA (US); Bryan Schellberg, Pawtucket, RI (US); Ryan Patrick Brady, Seattle, WA (US)

(21) Appl. No.: 19/125,925

(22) PCT Filed: Nov. 21, 2023
(86) PCT No.: PCT/US2023/080823

§ 371 Date: Apr. 30, 2025

Related U.S. Application Data
(60) Provisional application No. 63/384,887, filed on Nov. 23, 2022.
(60) Provisional application No. 63/385,152, filed on Nov. 28, 2022.
(60) Provisional application No. 63/509,103, filed on Jun. 20, 2023.

(51) Int. Cl. C12M 3/06; C12M 1/00; C12M 1/36; C12M 3/00

(52) U.S. Cl. CPC C12M 23/16; C12M 21/08; C12M 29/00; C12M 41/48

(57) ABSTRACT

Embodiments disclose a fluidic device. Said device has first and second layers that are configured to be coupled together via magnetic attraction to form a cavity. An embodiment defines an apical layer and a basal layer and a membrane therebetween within the cavity. Another embodiment of the fluidic device comprises a port positioned in optical arrangement with the internal cavity for optical observation of fluids or activities therein. These embodiments provide improvements to organ-on-chip (OoC) scalability, assembly and disassembly as well as cell observation with low disturbance to the culture inside the cavity.

### RELATED APPLICATIONS

This application claims the benefit of U.S. Provisional Application No. 63/384,887, filed on Nov. 23, 2022; U.S. Provisional Application No. 63/385,152, filed on Nov. 28, 2022; and U.S. Provisional Application No. 63/509,103, filed on Jun. 20, 2023. The entire teachings of the above applications are incorporated herein by reference.

### GOVERNMENT SUPPORT

This invention was made with government support under Grant Number GM142741 from the National Institutes of Health. The government has certain rights in the invention.

### BACKGROUND

Microfluidic devices, also known as “organ-chips,” or, “organ-on-chip,” (OoC) are an emergent technology that bridges a gap between in-vitro and in-vivo models used to investigate biological questions. These systems are bench-top devices containing miniature biological tissues for the purpose of study, research, and engineering. These devices integrate three-dimensional tissue architecture in-vitro to recapitulate organ-specific functions, such as liver metabolism and intestinal barrier function. In practice, these organ-chips can be used to reduce or eliminate a need for use of live animals in research. Because of this, there exists pressure from governmental agencies to advance microfluidics devices technology.

Existing methods suffer from scalability and reconfigurability issues. Further, although advances in OoC technology offer significant improvements to human-specific disease modeling and drug toxicity prediction, current platforms are hampered by complicated workflows and laborious offline analysis. Embodiments disclosed herein address these issues.

### SUMMARY

Disclosed herein is a fluidic device including a first layer having a first interface side and an opposing side, the opposing side defining a first complimentary portion of a cavity. The device has a second layer having a second interface side and an opposing side, the second interface side defining a second complementary portion of the cavity. The first and second layers are configured to be coupled together via magnetic attraction, the coupling of the layers mates the first and second interface sides together. The mating of the first and second interface sides forms a cavity, at least a portion of which is defined by the complementary portions, the first and second complementary portions in a coupled arrangement are configured to contain a fluid.

The fluidic device may also include one or more gaskets affixed to the interface sides and arranged to form a perimeter around the first complementary portion of the cavity and the second complimentary portion of the cavity.

The fluidic device may also include a third layer positioned between the first layer and second layer, the positioned third layer creating an upper channel in either the first layer or second layer, or a lower channel in either the first layer or second layer.

An embodiment of the fluidic device may also include internal microchannels defined at least in part by the cavity formed by the mating of the first and second interface sides. Also included is at least one valve operably coupled to either the first or the second layer, and operable from the opposing side of either the first or the second layer. The at least one valve is configured to redirect or block fluid flow between the internal microchannels.

The fluidic device may also include four edges each located perpendicular to the opposing side of the first layer and the opposing side of the second layer such that a rectangular prism is formed by the mating of the first interface side and the second interface side. As well as at least one interlocking joint positioned on an edge, the at least one interlocking joint configured to allow the fluidic device to be reconfigurably interlocked with an interlocking joint of another device.

An embodiment of the fluidic device may include a permanent magnet or an electromagnet coupled to, or captive in, the first layer and producing a magnetic attraction in combination with a metallic component coupled to the second layer, or in combination with a permanent magnet or electromagnet coupled to, or captive in, the second layer.

The previous embodiment may also include that the magnetic attraction has a flux density, and the flux density has a strength adjustably variable by a user.

Disclosed herein is a fluidic device including a structure defining at least one internal cavity configured to contain a fluid. In addition, at least one port defined by the structure and oriented in optical arrangement with the at least one internal cavity, the at least one port oriented in a direction enabling optical viewing into the cavity. The at least one port configured to couple an optical transmission channel to the fluidic device in a fixed position relative to the at least one cavity, the optical transmission channel configured to transmit optical radiation to or receive optical radiation from the fluid in the at least one cavity.

The fluidic device may also include at least one membrane layer internal to the structure that is positioned to create a plurality of internal cavities in the structure, each of the plurality of internal cavities having respective ports positioned in line with at least one respective cavity.

An embodiment of the fluidic device may include the fluid containing photoluminescent materials that are capable of being excited by light.

An embodiment of the fluidic device may also include at least one optical sensing device operably coupled to the at least one port configured to record excitations from the photoluminescent materials in the fluid.

In an embodiment, the structure, and at least one internal cavity defined therein, is configurable to operate as an Organ on Chip.

In an embodiment, the optical radiation transmitted to or received from the structure is automatically tuned based on a benchmark sample.

The fluidic device may also include the optical transmission channel and a single-mode wavelength filter element disposed within the optical transmission channel.

The fluidic device may also include the optical transmission channel and a multi-mode wavelength filter element disposed within the optical transmission channel.

The fluidic device may also include a plurality of ports positioned in optical arrangement with at least one of the at least one internal cavity. Each port of the plurality of ports is configured to receive a set of optical fibers arranged in a direction of a flow path defined by the at least one cavity, the optical fibers configured to record longitudinal changes of fluid flow over time in the flow path.

The fluidic device may also include gaskets positioned at the at least one port to seal the port fluidically.

The fluidic device may also include a processor operably coupled to the fluidic device and configured to process optical data collected as a function of a signal captured via the optical transmission channel.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a diagram of a scene that shows a representation of an embodiment in which a user is depicted sitting at a laboratory benchtop, performing an experiment on a fluidic sample using a reconfigurable organ-on-a-chip (OoC) device using a magnetic seal embodiment disclosed herein.

FIGS. 2A and 2B are mechanical assembly diagrams that show a top side and a bottom side of the OoC device according to the embodiment of FIG. 1.

FIG. 3 is a flow diagram of an example embodiment of a method for assembling an embodiment of the OoC device of FIG. 1.

FIG. 4 is a scalable mechanical assembly where several OoC devices are connected together utilizing interlocking joints so that they may be easily assembled and disassembled, according to an embodiment.

FIG. 5 is a top side of a 3D printed chip-top and OoC adhered together with integrated magnets, according to an embodiment.

FIG. 6 is a plot illustrating the magnetic flux density between magnets in relation to their distance from one another in the OoC, such as the OoC in FIGS. 2A-2B, FIG. 4 and FIG. 5, according to an embodiment.

FIG. 7 is an exploded view of an OoC in which several layers of different thickness and function are stacked atop of one another to create an OoC set up with integrated ports for optical fibers for fiber photometry, according to an embodiment.

FIG. 8 is a diagram of an example fiber photometry configuration for viewing photoluminescence in an OoC, according to an embodiment.

FIGS. 9A and 9B show illustrative views of the internals of an OoC which fiberoptics view into an apical (upper) layer, a membrane, and a basal (lower) layer, for viewing fluorophore material in each layer, according to an embodiment.

FIGS. 10A and 10B show an illustrative view of the internals of an OoC where fiberoptics view into an apical layer, a membrane, and a basal layer, for viewing fluorophore material in each layer. FIG. 10A shows a healthy cell monolayer, and FIG. 10B shows a damaged monolayer, according to an embodiment.

FIG. 11 is a generalized diagrammatic fiber photometry setup utilized in a long-term culture and sensing approach, according to an embodiment.

FIG. 12 is a representation of an OoC embodiment where the flow channels are staggered to allow for multiple chips to be utilized on a single block.

FIG. 13 is a plot representing the non-invasive sensing of fluorescein dextran on-chip over a period of ninety hours.

FIGS. 14A and 14B are plots representing a comparison of apparent permeability across fluorophore (dextran) size over a period of twenty hours.

FIG. 15 is a plot comparing the apparent permeability of a dextran size of 4 kDa and 70 kDa.

FIG. 16 is a plot representing the non-invasive sensing of fluorescein dextran concentration on-chip over a period of ninety hours.

FIG. 17 is a plot representing the non-invasive sensing of lucifer yellow across three chips simultaneously, over twenty-four hours using high-density fiberoptic cables.

### DETAILED DESCRIPTION

A description of example embodiments follows.

Organ-on-a-chip (OoC) devices present an exciting technology to improve an ability of scientists to mimic human biology on a bench-top. OoC devices are also referred to herein as “chips,” or “organ chips.” Advancements in OoC platforms have not been broadly adopted since OoC technology is difficult to scale and has not been reusable. Thus, OoCs have been difficult to use practically by the end customer. Legacy OoC systems have previously been constructed utilizing an adhesive compound to bind the layers of the OoC system permanently together. While effective, this method creates a single-use OoC.

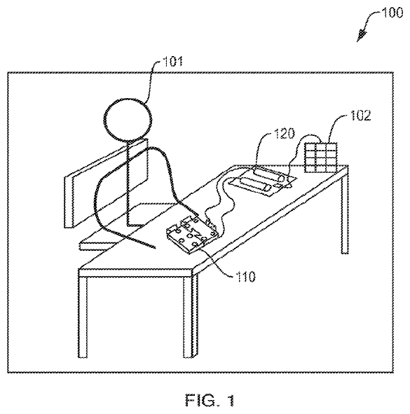

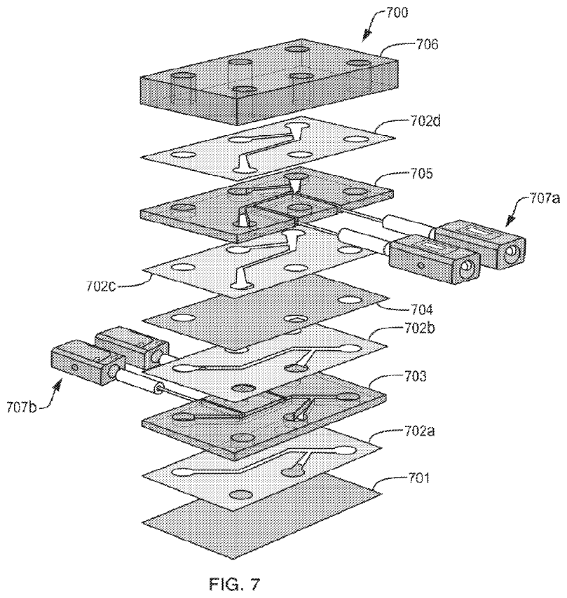

Embodiments described herein disclose a new method for sealing these chips in a reusable manner that allows rapid assembly and disassembly to improve usability. FIG. 1 through FIG. 6 relate to an embodiment of the present disclosure for a reconfigurable, magnetically adhered, organ on chip platform. Furthermore, embodiments described herein disclose a new method for fluorescence imaging. FIG. 7 through FIG. 17 relate to an embodiment of the present disclosure for a method of viewing, and recording, excited photoluminescent materials, such as fluorophores, within an OoC using fiber photometry.

Embodiments of the invention disclose a reconfigurable OoC by utilizing non-permanent methods of binding the layers of the OoC together. In particular, embodiments utilize magnets (such as neodymium magnets) to adhere the layers of the OoC together. This method allows for the OoC to be easily assembled, disassembled, reassembled, and reconfigured without having to create entirely new layers. The layers are fluidically sealed to prevent leaking via gaskets on the layers. The rapidity and ease of layer disassembly allows easy tissue capturing to allow high resolution imaging and molecular biological analysis (RNA seq, PCR, western blot). This allows for easy mid-experiment sampling and modification.

FIG. 1 is a representation of a scene 100 showing a user 101 sitting at a laboratory benchtop and performing an experiment on a fluidic sample using a reconfigurable organ-on-chip (OoC) device, also referred to herein as an OoC 110. The OC 110 shown in FIG. 1 is an example of a re-configurable embodiment. In this figure, two OoCs are shown interconnected; however, alternative embodiments may have one standalone OoC, or may have many OoCs interconnected. In this example embodiment, the OoC 110 is optically coupled to a fiber photometry set up 120, which is in turn connected to an LED driver 102.

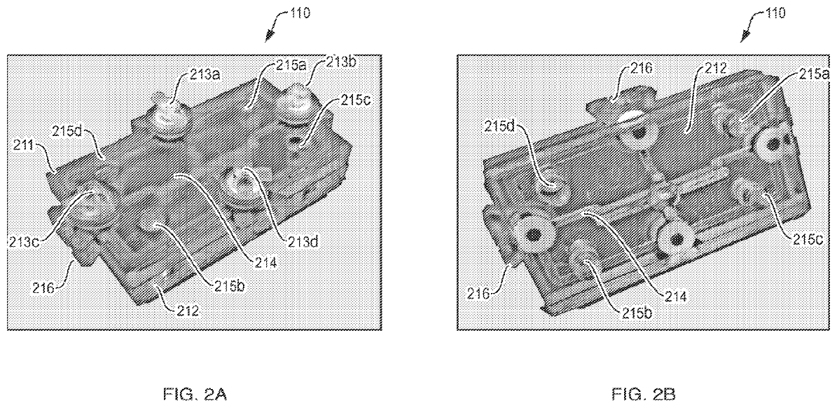

FIGS. 2A and 2B show a top side and a bottom side of the organ on-chip 110, respectively. The OoC 110 is constructed of a 3D printed chip top 211 interfaced with laser cut and assembled chip 212. The chip-top 211 and chip 212 may be adhered together using magnetic attraction, via magnets 215a-d. Laser-cut poly (methyl methacrylate) sheets are used to build the culture and flow compartments 214 of the chip 12, and stereolithography (SLA) 3D printed resin is used to build the chip top 211. The OoC 110 as shown has four valves 213a-d that are inserted with and sealed with fluoroelastomer o-rings to redirect or block fluid flow between the internal microchannels 214. Interlocking joints 216 are printed along with the chip tops and bottom. These interlocking joints 216 allow for the OoC to be set up or reconfigured within minutes. The reconfigurable chip orientation coupled with the integrated valves enable complete dynamic flow control without a need to adjust offline components.

Common in-vitro methods lack complexity required to recapitulate microenvironments of interest accurately, relying on static, two-dimensional paradigms to model dynamic, three-dimensional pathologies. Established in-vivo models offer improved complexity, but only elicit a subset of possible symptoms, often falling short in human-specific diseases [1, 2, 3]. In the context of drug discovery, translation from in-vivo to success in human clinical trials is abysmal, with failure rates of over 90% [4, 5]. Additionally, growing pressure from national agencies, including the EPA and Dutch NCad, to eliminate the use of animals in research limits future applicability of in-vivo studies [6, 7].

Over the past 30 years, OoC devices have emerged as a robust alternative to address technological gaps associated with current options [8]. OoC models integrate three-dimensional tissue architectures in-vitro to recapitulate organ-specific functions, such as liver metabolism and intestinal barrier function, enabling controlled interrogation of human-specific disease states [9, 10, 11]. These systems offer improved bio-relevance and controlled complexity via integration of physical and chemical stimuli matched to physiologically relevant conditions. For example, previous OoC platforms have implemented steep oxygen gradients to match physiological conditions in the small intestine and demonstrated the recovery of hepatocyte function in response to fluidic shear stress [12, 13].

Additional embodiments described herein disclose a new method for fluorescence microscopy. FIG. 7 through FIG. 17 relate to an embodiment of the present disclosure for a method of viewing, and recording, excited photoluminescent materials, such as fluorophores, within an OoC using fiber photometry.

A wide range of integrated sensing on-chip, including electrochemical and optical modalities, capable of sensing transient cellular responses in-situ exist, however, most are expensive, single use, semi-destructive, and susceptible to fouling

Embodiments disclose an OoC platform that enables automated, spatiotemporal data collection for long-term characterization of living cell culture conditions. These embodiments may be used to assess barrier function of epithelial and endothelial tissue, as well as metabolic function and calcium flux in two- or three-dimensional cell cultures. This is accomplished by integrating noninvasive optical sensing modalities and automated controls into a multilayer organ-chip. Furthermore, use of optical tools for sensing biological systems is a rapidly growing field. However, utilization of this technology has not been translated to experiments in dish or OoC tools.

Utilizing these embodiments of an OoC with integrated optical sensing modalities, and by adding fiber optics into the system, light can be precisely delivered to the samples in the OoC, and emissions from samples can be recorded. This ability opens the OoC platform up for a wide range of live in-situ measurements including, but not limited to, transport of fluorescently labeled molecules, cell activity via calcium flux, and expression of tagged proteins. This ability makes embodiments greatly more impactful and user-friendly, allowing non-contacting, real-time monitoring of cell function.

Embodiments describe a number of methods to fabricate bonded, thermoplastic OoC platforms that can be designed or machined to include a number of ports. These ports can be utilized to insert ferrule coupled fiber optic stubs (strengthened fiber optic connection) into the OoC platform. Using a ferrule-ferrule coupling, OoC chips can be connected to an optics assembly that includes, for example, an LED light source for excitation and a CMOS camera for recording emissions. Appropriate dichroic mirrors and wavelength filters can be easily swapped for specific fluorescent proteins and reporters. Within the OoC, optical fiber-based sensing allows for a broad range of bioluminescent assays, including concentration measurements, expression profiles, and calcium sensing.

Referring now to embodiments relating to a new method for sealing OoC chips in a reusable manner which allows rapid assembly and disassembly to improve usability (FIG. 1 through FIG. 6), laser-cut poly (methyl methacrylate) sheets are used to build the culture and flow compartments of the chip, and stereolithography (SLA) 3D printed resin tops provide the described functionality. The chip top features joints that interlock, like puzzle pieces, with up to four independent 3D printed chip connectors. Joints between tops and connectors fit together with a simple “push-to-fit” action, enabling rapid setup and reconfiguration of scalable chip arrays. Internal microchannels pass through the center plane of both the top and connector, allowing fluid flow from one chip to another via in/outlets at each of the joint faces. Fluid flow within, and across, chips is directed using 3D printed hand-operated valves that enable flow path customization.

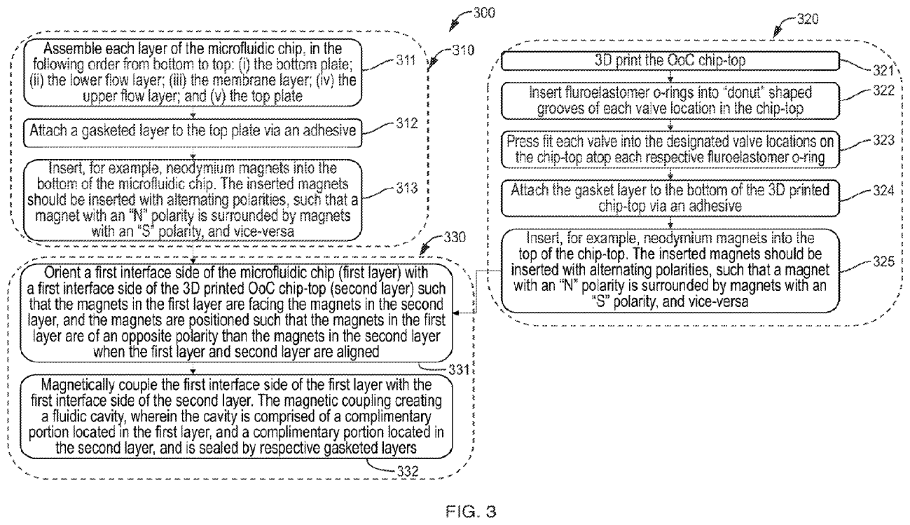

FIG. 3 is a flow diagram of an example embodiment of a method 300 that includes submethods 310 (assemble layers), 320 (apply magnets), and 330 (couple layers via magnets), respectively, for assembling an embodiment of the OoC device of FIG. 1. Both the microfluidic chip and the 3D printed OoC are constructed separately, then combined together. The method 300 to assemble the microfluidic chip. The assembler starts with the first submethod 310 by assembling each layer of the microfluidic chip, in the following order from bottom to top: (i) the bottom plate; (ii) the lower flow layer; (iii) the membrane layer; (iv) the upper flow layer; and (v) the top plate. Next, the assembler attaches 302 a gasketed layer to the top plate via an adhesive. The assembler then inserts 303, for example, neodymium magnets into the bottom of the microfluidic chip. The inserted magnets are inserted with alternating polarities, such that a magnet with an “N” polarity is surrounded by magnets with an “S” polarity, and vice-versa.

Still referring to FIG. 3, the assembler in the submethod 310 builds the 3D printed OoC chip-top. In an embodiment, the chip-top may be 3D printed 321. The assembler is then able to insert 322 the fluoroelastomer o-rings into “donut” shaped grooves of each 3D printed valve location in the 3D printed chip-top. The assembler may press fit 323 each valve into the designed 3D printed valve locations, atop each respective fluoroelastomer o-ring. Next, the assembler attaches 324 the gasket layer to the bottom of the 3D printed chip-top via an adhesive. The assembler finishes the chip-top assembly by inserting 325, for example, neodymium magnets into the top of the chip-top. The inserted magnets are inserted with alternating polarities, such that a magnet with an “N” polarity is surrounded by magnets with an “S” polarity, and vice-versa.

Still referring to FIG. 3, assembler in the third submethod 330 creates the microfluidic chip. The third submethod 330 combines the products of the first and second submethods 310 and 320, according to an embodiment. The third submethod 332 begins by orienting 331 a first interface side of the microfluidic chip (first layer) with a first interface side of the 3D printed OoC chip-top (second layer) such that the magnets in the first layer are facing the magnets in the second layer, and the magnets are positioned such that the magnets in the first layer are of an opposite polarity than the magnets in the second layer when the first layer and second layer are aligned. The assembler's next step 322 magnetically couples 332 the first interface side of the first layer with the first interface side of the second layer. The magnetic coupling creates a fluidic cavity, wherein the cavity is defined by a complimentary portion located in the first layer, and a complimentary portion located in the second layer, and is sealed by respective gasketed layers. The coupled first and second layers create a magnetically sealed organ-on-chip platform for rapid assembly and disassembly.

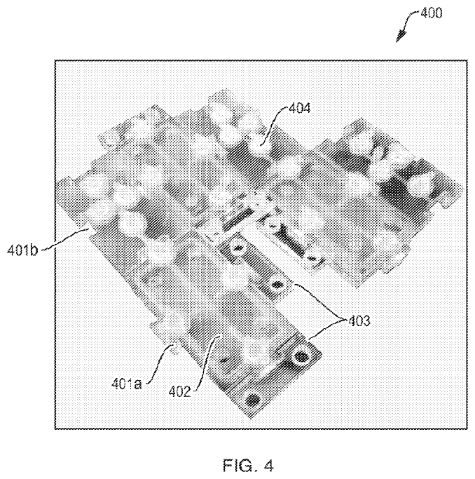

FIG. 4 shows a scalable chip embodiment 400 which demonstrates how interlocking joints 401a (male joint example) and 401b (female joint example) can be used to assemble multiple OoCs easily into one scalable chip array 400. The interlocking joints 401a-b between the tops 402 and connectors 403 fit together with a simple “push-to-fit” action, enabling rapid setup and reconfiguration of scalable chip arrays. The joints 401a-b interlock like puzzle pieces, with up to four independent 3D printed chip connectors 403. Internal microchannels pass through the center plane of both the top and connector, allowing fluid flow from one chip to another via in/outlets at each of the joints. Fluid flow both within and across the chip is directed using the 3D printed hand-operated valves 404, which enable flow path customization.

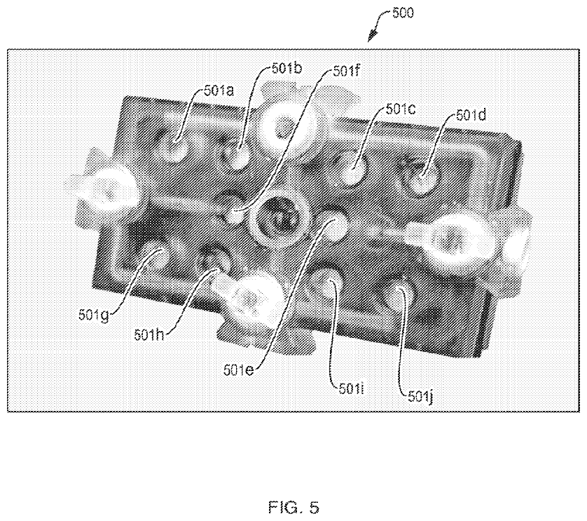

FIG. 5 shows an embodiment 500 in which the 3D printed top, and OoC, are adhered with integrated magnets according to an embodiment. This embodiment utilizes magnets 501a-j to adhere the layers of the OoC together. The use of the integrated magnets allows for the OoC to be easily assembled, disassembled, reassembled, and reconfigured without having to create entirely new layers. While permanent magnets are shown in FIG. 5, it should be understood that a variety of forms of magnetic attraction are acceptable. For example, alternative embodiments may be done via electromagnets. This embodiment may use a magnet attracted to a ferrous metal, a magnet attracted to a magnet, an electromagnet attracted to a ferrous metal, etc. In addition, the force of magnetic attraction may be appropriate enough in the case that the 3D printed top and the OoC are properly secured, such that the gasket between them sufficiently compresses to create a fluidic seal. In this embodiment, the magnetic flux is not so great so as to affect results of an experiment; although, it may be possible depending on the magnets used to tune the magnetic flux density for optimal results.

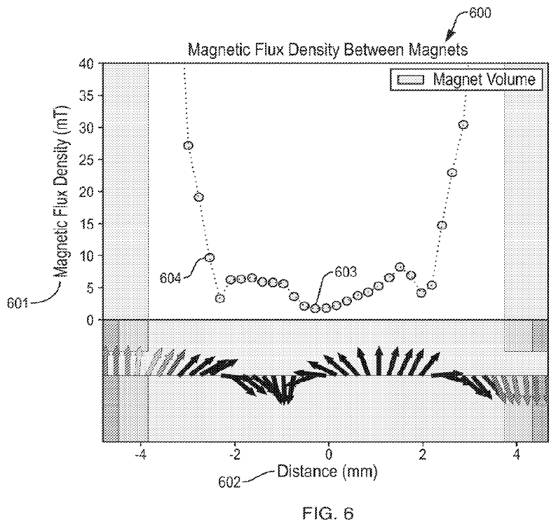

FIG. 6 shows a plot 600 illustrating magnetic flux density between magnets in relation to their distance from one another in the OoC, such as the OoC in FIGS. 2A-2B, FIG. 4, and FIG. 5. The plot 600 compares magnetic flux density in milli-Teslas (mT) 601 on the Y-axis, against relative distance 602 on the X-axis. This plot 600 is a computational model of the magnetic flux density across the cell culture area of an OoC. Magnetic flux density magnitudes range from 2 mT 603 to 10 mT 604, and are directionally disordered. In this configuration, the flux, strength, and orientation of the magnetic field will not significantly impact a cell culture.

Microfluidic devices are typically made of either polydimethylsiloxane (PDMS) which has a number of disadvantages (gas permeability, reliance on clean room, difficult to use) or bonded thermoplastics that limit a configuration of tissue organization and post culture analysis. The ease of layer disassembly disclosed by this embodiment allows easy tissue capturing for high resolution imaging and molecular biological analysis (RNA seq, PCR, western blot). This embodiment allows easy mid-experiment sampling and modification that is challenging with current strategy.

Further, the layer-by-layer assembly allowed by this embodiment allows a broad design freedom for the customer. For example, rapid assembly and disassembly with the magnetically sealed platform allows complex tissue processing and molecular biology techniques that are not possible with other strategies.

Referring now to embodiments for adapting fiber photometry as a noninvasive, automated optical sensing tool applied to OoC (FIG. 7 through FIG. 17), real-time, online readouts of luminescence intensity, pH, and oxygen concentration may enable correlation to relevant physiological parameters in a temporal manner, tracking transient cellular activity and response to perturbation in-situ. The addition of high-density fiber arrays allows spatial mapping of the whole-chip environment, and, when combined with real-time readouts, comprehensive tracking of cellular dynamics across the experimental timescale. With high-density fibers, whole-chip spatial sensing may be achieved across multiple OoC for high throughput data collection across samples. The fiber photometry embodiments may be further optimized for closed-loop, semi-automated culture maintenance, enabling data collection and analysis with limited human intervention. A semi-automated culture and sensing platform would significantly reduce required human interaction, and therefore limit potential procedural errors. Additionally, closed-loop culture maintenance may significantly improve long-term cell viability by continuously adjusting culture conditions to optimal conditions. This would have significant implication in modeling chronic or long-term pathology progression directly in OoC. In an example embodiment, fiber photometry embodiment is integrated with OoC to enable long-term spatiotemporal characterization of cellular activity and response to perturbation in a semi-automated and high throughput manner.

FIG. 7 shows an exploded view of an embodiment of an OoC 700 of the invention in which several layers are stacked to create an OoC setup for fiber photometry with integrated optical fibers. The OoC 700 is constructed utilizing a glass coverslip 701, double sided tape 702a-d, a lower flow channel 703, a membrane 704, an upper flow channel 705, and the OoC top 706. It should be understood that, while double sided tape 702a-d is used to adhere layers of the chip together, utilization of magnetic attraction may be used in the alternative to obtain similar results. The OoC 700, contains at least one internal cavity (e.g., the upper or lower flow channel) configured to contain a fluid. Fiber stubs 707a enter the OoC at a port defined by the upper flow channel 705, and fiber stubs 707b enter the OoC at a port defined by the lower flow channel 703. These ports may be oriented in an optical arrangement with the cavity and may enable optical viewing into the cavity. The fiber stubs 707a-b are reversibly inserted into laser-cut guide channels for optical access to cell culture channels. The fiber stubs 707a-b may be connected to an optical transmission channel configured to transmit optical radiation to, or receive optical radiation from, the fluid in the cavity.

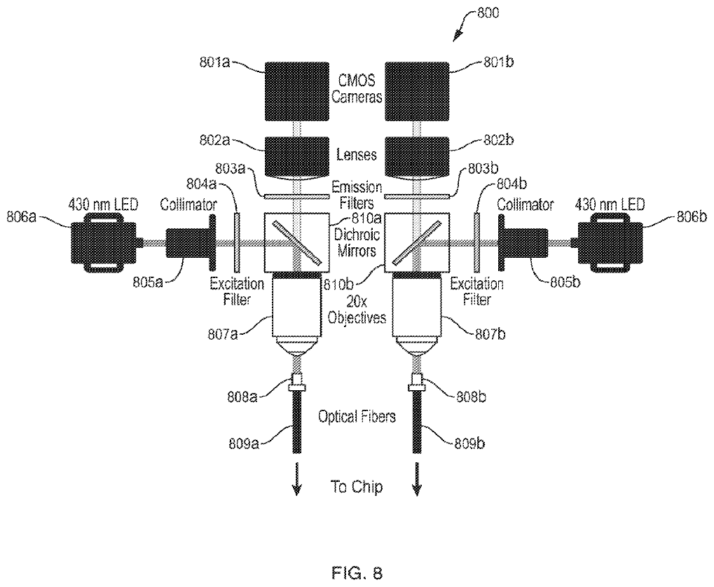

FIG. 8 shows an example layout of a fiber photometry setup 800. CMOS cameras 801a-b look through lenses 802a-b and emission filters 803a-b. LEDs 806a-b emit a respective light beam, which passes through collimators 805a-b and excitation filters 804a-b, respectively. The dichroic mirrors 810a-b direct the respective beam through 20× objectives 807a-b, which are connected to the fiber connectors 808a-b, which are connected to the optical fiber 809a-b. In an embodiment, the optical fiber 809a gets connected to the fiber stubs 707a (the upper flow channel, or apical layer), and the optical fiber 809b gets connected to the fiber stubs 707b (the lower flow channel, or basal layer). In an embodiment, LEDs 806a-b are driven by a 4-channel LED driver.

The photometry embodiments implement, and validate, fiber photometry (fiber optics) as a noninvasive and semi-automated sensing modality that enables spatiotemporal characterization of both the cellular microenvironment and function in OoC. Fiber photometry has broad applicability to all luminescence and potential for automated, high-throughput use. As an optical sensing modality, fiber photometry is less invasive, more robust, and has high reusability compared to electrochemical sensing approaches [16]. An integrated fiber photometry platform has the potential to elucidate transient cellular dynamics that have not previously been possible in a semi-automated and high-throughput manner, allowing for a shift away from laborious, bulky, and expensive microscopy or electrochemical characterization techniques.

Embodiments integrate fiber photometry into laser-cut and assemble multilayer organ-chips to enable real-time and spatially resolved fluorescence, oxygen, and pH sensing [26]. A fiber photometry platform may be constructed based on published protocols and further engineered for direct application in OoC [23, 25]. An engineering focus on integrating and validating sensing technologies in OoC may be conducted using cell-free samples in the organ-chip platform. Further validation of OoC device development will be applied to biological models on-chip.

An embodiment discloses an integrated fiber photometry system for real-time, in-situ tracking of fluorescence intensity on-chip. Recorded fluorescence intensity values are correlated to relevant model-specific readouts (e.g., concentration, Ca²⁺ flux). High-density fiber optic arrays may be implemented for spatially resolved recordings on-chip and simultaneous data collection from multiple OoC. Up to 48 independent optical fibers may record emitted fluorescence within and across OoC for high-throughput spatial sensing on-chip. Optical oxygen and pH sensing modalities may be added to the platform to characterize the cell culture microenvironment and assess cellular metabolism.

Spatiotemporal monitoring of epithelial membrane permeability may be conducted on-chip to validate fluorescence sensing, confirmed in parallel via live fluorescence microscopy and aliquoted culture media samples. Epithelial permeability may be compared across healthy and diseased models to ensure that expected responses are elucidated from data collected in-situ. The disclosed OoC platform may be extended to multimodal (fluorescence, oxygen, pH) sensing and characterization of cytosolic Ca²⁺ flux in human neural stem cells transfected with GCaMP8. Ca²⁺ flux may be used to assess neuronal firing frequency and amplitude under control and stimulation conditions, enabling noninvasive interrogation of cellular activity in-situ.

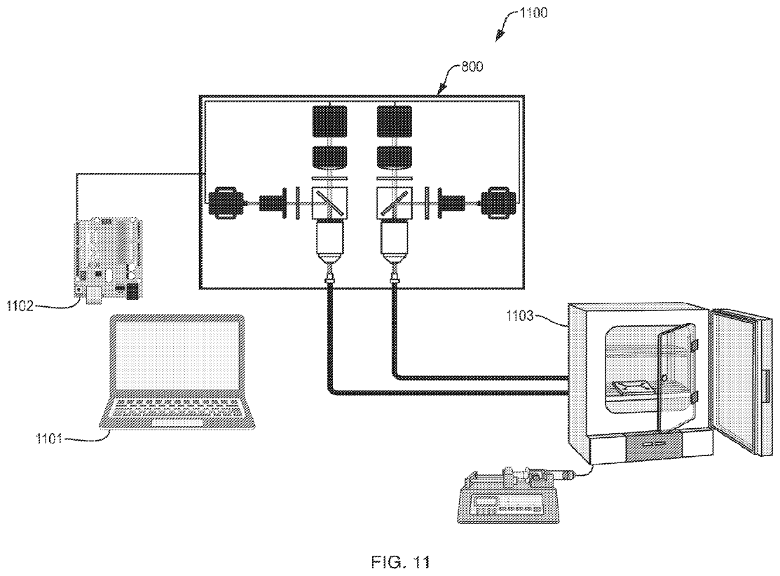

Fluorescence, oxygen, and pH signals sensed via integrated fiber photometry may be recorded in real-time by image capture triggers delivered by a microcontroller (as shown in FIG. 11). A data analysis pipeline may automate analysis of collected multimodal, spatiotemporal data to enable real-time streaming of relevant cell culture conditions. Simultaneous collection of functional outputs of cell health may be used to correlate environmental perturbations to observed responses on-chip. The controller may be a proportional-integral-derivative (PID) controller that may be engineered from observed cellular responses and integrated on-chip for closed-loop maintenance of cell culture conditions. Automated data acquisition and analysis with integrated controls may be validated over long-term culture (e.g., months) of human neural stem cells, or similar long-lived cell types. The disclosed photometry platform may be used to sense responses in Ca²⁺, pH, and oxygen concentration to periodically scheduled perturbations throughout culture. Cell viability may be assessed throughout the culture period to determine effectiveness of closed-loop control on-chip compared to steady flow.

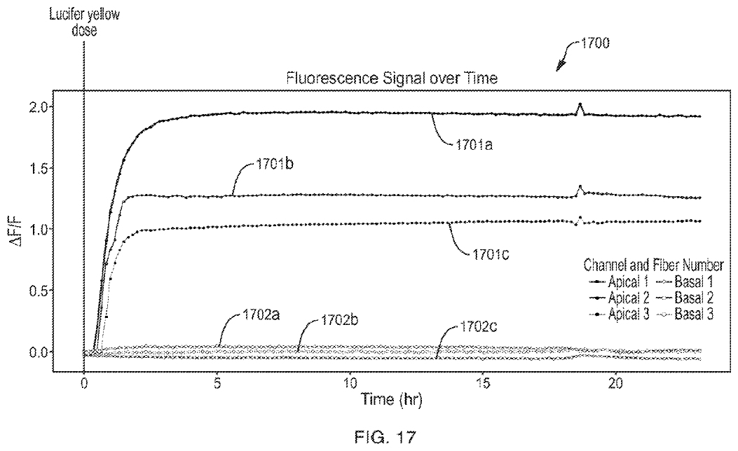

Fiber optic cables may be readily interfaced with OoC platform via engineered fiber guides. Referring to FIG. 7, laser-cut guides fit to the dimensions of commercially available fiber stubs 707a-b enable reversible insertion of fiber ends into the OoC for near-direct contact with the cell culture channels 703 and 705. A 500-micron poly-methyl methacrylate (PMMA) wall separates the fluidic culture chamber from the fiber tip, allowing for noninvasive sensing with minimal attenuation [27]. Lucifer yellow (428 ex./536 em.) may be detected on-chip in a concentration dependent manner as demonstrated in FIG. 17. Constructing a calibration curve confirms that the platform detects changes in fluorophore concentration and that observed trends match microplate reader outputs from collected sample effluent. Embodiments may have luminescence independent sensing capability. Concentration dependent fluorescence sensing may be achieved using fluorescein dextran (494 ex./521 em.) following an identical workflow to lucifer yellow with minor adjustments. To adapt the embodiment for fluorescein dextran sensing, the excitation filter (430 center wavelength (CWL) to 490 CWL) and fiber coupled LED (430 nm to 470 nm) may be exchanged to account for wavelength differences across fluorophores. The sensing approach may be applicable in three-dimensional (3D) culture systems, sensing dispersed fluorescent polystyrene beads encapsulated in fibrin hydrogel at varied bead-gel weight ratios.

High-density fiber optic arrays may be implemented for spatially resolved, whole-chip recordings. High-density fiber photometry recordings and spatial mapping has previously been demonstrated in freely moving animals to record calcium flux across murine brain structures but has yet to be applied in-vitro or on-chip [22]. Initial high-density recordings may be validated using a 1-to-7 fan-out fiber bundle to record at seven distinct locations lining the on-chip flow path. The fan-out fiber bundle splits the optical path across seven individual fibers, each capable of interfacing with OoC as described for a single fiber previously. A bolus of fluorescent dye may be injected on-chip and tracked by the integrated fibers along the laminar flow path, cross validated with fluorescence microscopy. To achieve higher spatial fidelity, either commercially available fiber optic ferrules capable of holding up to 48 discrete fibers, or 3D printed custom ferrules may be integrated into the optical beam path. Connector ports to interface the ferrules with OoC may be engineered using CAD software and built via laser-cut multilayer assembly or by 3D printing. In some embodiments, convex cylindrical lenses may be used to shape the beam onto the objective focus plane to record from all fiber channels simultaneously. A high-density system that includes at least 24 discrete optical fibers may be validated on-chip by the identical methods described for the fan-out fiber bundle.

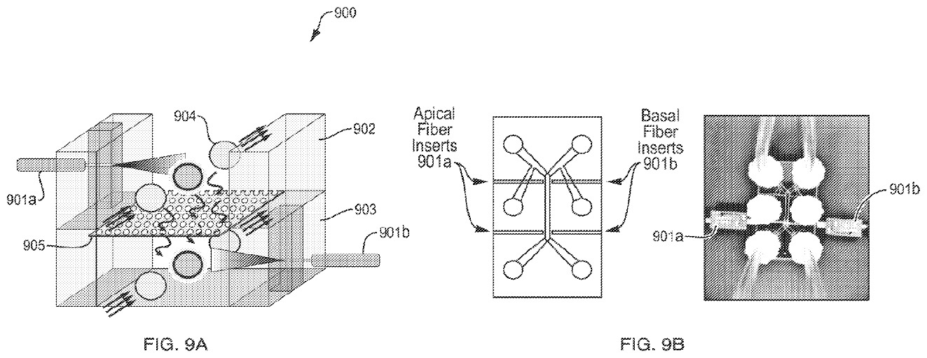

FIGS. 9A and 9B show illustrative views of internals of an OoC 900 in which fiberoptics view into an apical layer, a membrane, and a basal layer, for viewing fluorophore material in each layer. Fiber optical cables 901a-b reach into the organ chip in both the apical layer 902 and the basal layer 903. In this embodiment, a diffusion of fluorophore 904 can be seen as it passes from the apical layer 902 through the semi-permeable membrane 905 into the basal layer 903.

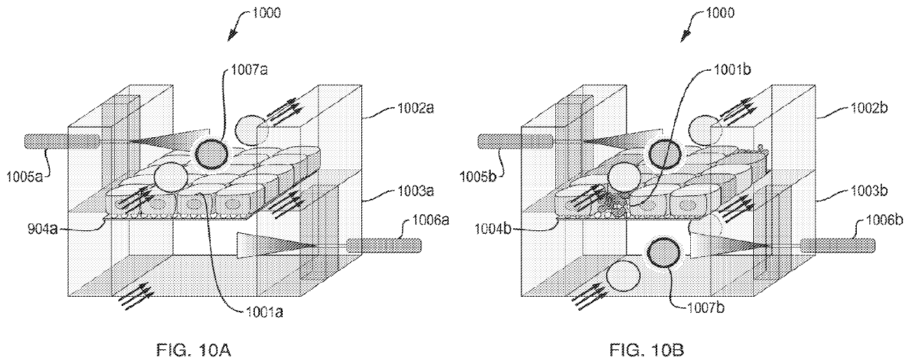

FIGS. 10A and 10B show an example embodiment 1000 in which the fluorophore concentrations 1007a-b are monitored across the upper 1002a-b and lower 1003a-b channels of an OoC meant to resemble a gut setting. FIG. 10A shows the OoC with a healthy monolayer 904a, no fluorophore 1007a should be detected in the lower channel 1003a. If the monolayer is disrupted, diffusion of the fluorophores through the semi-permeable membrane 1004a will be detected. FIG. 10A shows that the monolayer 1001a is healthy, and as such there is no fluorophore in the basal layer 1003a. However, in FIG. 10B, it is shown that the monolayer 1001b is damaged, and as such there is fluorophore 1007b diffusing through the semi-permeable membrane 1004b from the apical 1002b layer to the basal 1003b layer. Optical fibers 1005a-b allow data to be transmitted from the apical layers 1002a-b; and optical fibers 1006a-b allow data to be transmitted from the basal layers 1003a-b.

Optical oxygen and pH sensing modalities may be added in conjunction with fluorescence sensing for multiplexed assessment of cellular activity, acidification, and cellular respiration. Real-time monitoring of respiration (oxygen) and acidification (pH) rates offers direct quantification of cellular metabolism to determine responses to microenvironment perturbation. Sensing may be achieved via fiber photometry and fluorescent probes, and initially validated using organ-chip systems containing no biological samples. Oxygen sensitive probes, such as ruthenium dyes, are quenched in the presence of molecular oxygen and changes in fluorescence intensity may be correlated directly to concentration [16,30]. To achieve oxygen sensing on-chip, ruthenium dye may be added to the culture medium and emitted fluorescence will be monitored as a real-time readout of oxygen concentration. Oxygen sensors may be calibrated via two-point calibration at anoxic and air-saturated conditions in organ-chips containing cell culture medium only. An oxygen scavenging compound, such as sodium sulfite, may be added to culture medium to achieve initial anoxic conditions and maintained by the disclosed gas-impermeable organ-chip design [31]. Real-time pH sensing may be achieved by measuring the absorbance of a pH indicator, such as phenol red, within the cell culture media or from an embedded sensor spot [31, 32]. If necessary, an additional reference dye, such as Egyptian Blue, may be added to the medium or spot for dual lifetime referencing to determine pH values [31]. The pH sensor may be calibrated in a blank organ-chip by stepwise increase in pH of a buffer solution ranging from 5-9. For both oxygen and pH, 2-6 discrete optical fibers from a high-density ferrule may be retrofitted and integrated. The resulting sensing platform will enable noninvasive, spatiotemporal monitoring of fluorescence intensity, pH, and oxygen concentration.

The fiber photometry embodiment has been validated for in-vivo optical recordings, and results from preliminary work suggest robust luminescence sensing on-chip [21, 22, 23]. Multiple luminescent species with minimal peak wavelength overlap may be implemented to extract independent fluorescence, oxygen, and pH signals. Photomultiplier tubes may be integrated into the embodiment to increase emission signal to a detectable range or to improve signal-to-noise ratio. Microscopy may be conducted to validate embodiment functionality across all sensed parameters.

Embodiments demonstrate fluorescence tracking of epithelial and endothelial monolayer permeability and multimodal (fluorescence, oxygen, pH) sensing of cytosolic Ca²⁺ flux in human neural stem cells transfected with GCaMP8, a fluorescent calcium reporter.

The integrated fiber photometry embodiment may perform as a real-time optical sensor of epithelial barrier function. Caco-2, a human colorectal adenocarcinoma cell line, may be cultured on-chip to model the epithelial monolayer, and lucifer yellow dye may be used to track permeability based on established protocols [26]. Although current permeability assays provide valuable insight into the overall barrier function of the system, such assays are typically limited to time-point sampling of fluorophore concentration, limiting the potential to observe transient responses to perturbation [33, 34]. At least some of the embodiments of the invention an approach to track barrier function in real-time, at least temporarily via integrated fiber photometry through the use of the organ-chip platform.

FIG. 11 shows an embodiment of the fiber photometry setup 800 utilized in a long-term culture and sensing approach 1100. An external laptop 1101 and a microcontroller 1102 deliver triggers to and collect data from the fiber photometry platform 800. Optical fibers fed through an incubator 1103 enable long term sensing.

The microcontroller 1102 may deliver electronic, transistor-transistor logic (TTL), triggers to capture images and store outputs from may be employed to align fiber photometry platform to an external computer. A custom microcontroller script aligns LED pulses with CMOS camera capture triggers to collect discrete timestamped data from the on-chip microenvironment during fluorophore excitation and emission. Time between LED pulses, pulse width, and delay between excitation and camera capture may be taken into account to optimize data capture to maximize data quality depending on the culture model.

Data collected via fiber photometry may be analyzed in real-time with commercial off-the-shelf or customized software or data processing platforms, also referred to herein as an “analysis tool,” to identify absolute fluorescence intensities and track intensity changes as a function of time and on-chip location. Images captured by the CMOS (or other technology) camera may be automatically read by an analysis tool to provide real-time signal readout. Continuous data analysis enables feedback control by tracking deviations from baseline cellular activity in real-time and without user intervention.

An embodiment for semi-automated maintenance of cell culture conditions may be validated over culture periods up to at least three months depending on lifespan of the cell culture. Baseline Ca²⁺ flux, oxygen concentration, and pH data may be used as setpoint values and maintained via closed-loop PID control by comparison to data collected on-chip in real-time. Scheduled perturbation on-chip may be used for initial optimization of control parameters and to characterize overall platform robustness. Cell viability may be assessed throughout extended culture periods by observed Ca²⁺ flux, cross validated by fluorescence microscopy and live/dead assays [39]. The effectiveness of closed-loop control on-chip may be compared to steady flow controls by measured cell viability, metabolic activity, and Ca²⁺ flux.

Intact Caco-2 monolayers may be characterized to determine baseline permeability. Dextran sodium sulfate (DSS), a compound commonly used to induce colitis in-vivo, may be dosed on-chip to disrupt the Caco-2 monolayer integrity [35]. Transient changes in monolayer permeability may be tracked prior to DSS dosing, during exposure, and post-dosing. Data collected in-situ may be compared across control and diseased models to verify that expected responses are elucidated by fiber photometry. Outputs from fiber photometry may be cross validated by comparison to live fluorescence microscopy and to aliquoted samples measured by microplate reader.

To validate embodiments of the fiber photometry platform for multimodal, spatiotemporal sensing of fluorescence, oxygen concentration, and pH, tracking of the cellular activity of human neural stem cells on-chip may be performed. Human neural stem cells transfected with the genetically encoded calcium indicator GCaMP may be encapsulated in fibrin hydrogel and seeded on our organ-chips. Cytosolic Ca²⁺ flux, oxygen concentration, and pH may be observed under control conditions to determine baseline flux and metabolic activity [22]. Potassium chloride may be dosed on chip to induce membrane depolarization and increase Ca²⁻ flux as a stimulation condition [38]. Ca²⁺ flux, recorded via fluorescence intensity changes in GCaMP signal, may be used to assess and compare neuronal firing frequency and amplitude under control and stimulation conditions, enabling noninvasive interrogation of cellular activity in-situ [21,22]. Oxygen concentration and pH may be recorded to determine the effect of potassium chloride, if any, on neuron metabolism. The effect of cell seeding density may also be investigated to determine an optimized seeding density that maximizes GCaMP signal and minimizes spatial gradients in metabolic activity resulting from nutrient consumption.

To demonstrate the broad applicability of fiber photometry as a real-time sensor of barrier function, an additional, orthogonal cell line may be used. Human umbilical vein endothelial cells (HUVECs) may be used to model the permeability of endothelial monolayers on-chip following protocols adapted from the literature [36, 37]. A similar validation and experimental workflow may be used as described for Caco-2, with optimization of DSS concentration or use of an alternative compound depending on cellular sensitivity. Transient effects on monolayer permeability may be analyzed and compared to results from Caco-2 experiments to validate fiber photometry as a real-time optical sensor of barrier function independent of cell type. Cardiomyocytes labeled with the intracellular calcium reporter Fluo-4 may be used as an alternative validation of multimodal sensing of Ca²⁺ flux, oxygen concentration, and pH on-chip.

Real-time data streaming via fiber photometry may be leveraged to integrate closed-loop control on-chip, enabling semi-automated maintenance of cell culture conditions over extended times. Dynamic maintenance of cell metabolism has the potential to extend culture lifetimes, delivering nutrients and removing waste products at rates tailored to real-time cellular activity. Fiber photometry outputs of fluorescence, oxygen concentration, and pH may be used as control parameters, fed to a PID controller in real-time to maintain cell viability over culture times up to three months.

Delivered TTL (or other electrical signal) trigger parameters may be optimized to maximize fluorophore emission and minimize photobleaching. Robust image analysis packages will be used to analyze captured data and results may be verified by an analysis tool. The feedback control system may be reduced to open loop control for more consistent input management. Direct readouts and suggestions may be provided to the user to maintain device ease of use.

A cell-free organ-chip system may first be implemented to characterize the fiber photometry system and optimize sensing parameters. For initial platform validation, fluorescence microscopy and timepoint absorbance measurements may be conducted in parallel to confirm observed readouts. On chip Caco-2 and human neural stem cell cultures may be validated based on established cellular responses and phenotypes. Caco-2 cells may be characterized functionally by previously established membrane permeability assay and visually by positive immunostaining. Human neural stem cell function may be characterized by live calcium imaging and cell structure will be validated by positive immunostaining.

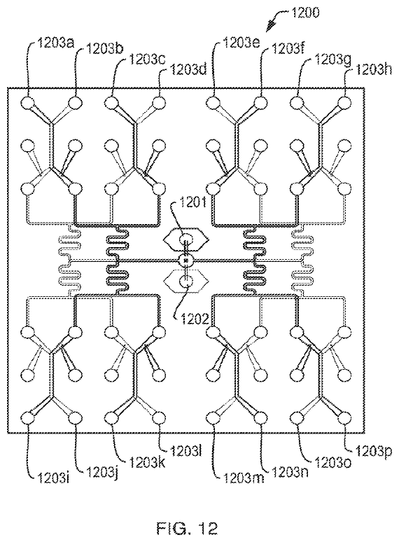

FIG. 12 shows a representation of an OoC embodiment 1200 in which the flow channels are staggered to allow for multiple chips (not shown) to be utilized on a single block. This particular example allows for two inlets, one to the apical layer 1201 and basal layer 1202, as well as independent outlets 1203a-p each from the apical and basal layers. In this staggered configuration, there are no overlapping channels, and each outlet is in a separate layer with a PET barrier. An embodiment such as the one of FIG. 12 allows for equal flows across channels, prevents backflow from one outlet to another, and allows for streamlined chip assembly.

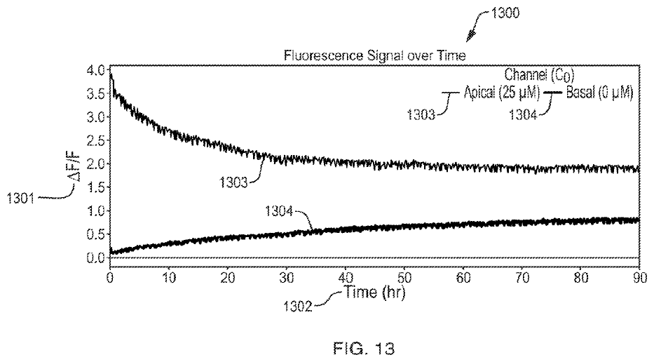

FIG. 13 shows a plot 1300 representing the non-invasive sensing of fluorescein dextran on-chip. The plot 1300 shows the fluorescence signal over time by plotting the change in fluorescence intensity 1301 (where (ΔF/F=(F−Fᵣₑₛₜ)/Fᵣₑₛₜ)) over time 1302 in the apical layer 1303 (25 μM channel) and in the basal layer 1304 (0 μM channel). This plot 1300 shows the sensed fluorescence intensity from the apical 1303 and basal 1304 organ-chip channels over a period of four days. The diffusion of 70 kDa fluorescein dextran from the apical channel to the basal channel was observed as a respective decrease, and consequential increase, in sensed fluorescence intensity. These measurements were recorded under static conditions.

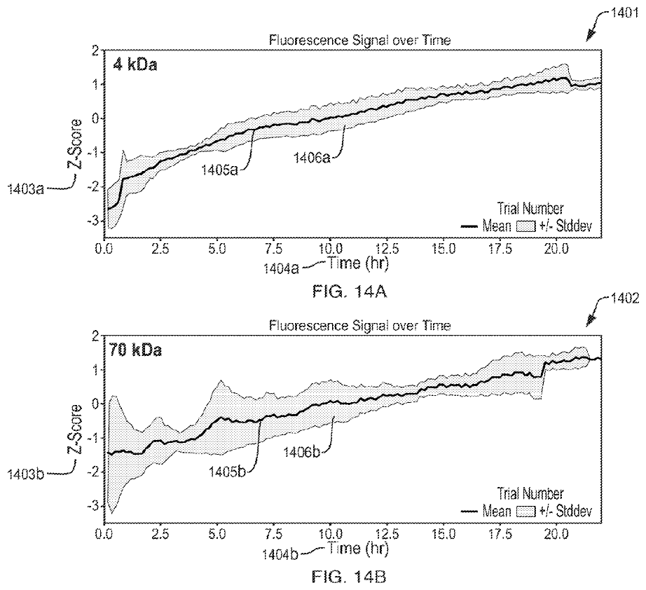

FIGS. 14A and 14B show plots 1401 and 1402 representing a comparison of apparent permeability across fluorophore size. Plot 1401 represents results with 4 kDa and plot 1402 represents results with 70 kDa. Both plots 1401 and 1402 show the fluorescence signal over time by plotting the z-score 1403a-b over time 1404a-b. Both plots 1401 and 1402 plot a mean trial number 1405a-b against a standard deviation 1406a-b. The mean±standard deviation of sensed fluorescence z-score for 4 kDa and 70 kDa fluorescein dextran over a 24-hour period. An increasing z-score shows increasing basal concentration of fluorescein dextran due to diffusion. Data represents sensed fluorescence from the basal channel only across five independent trials per condition.

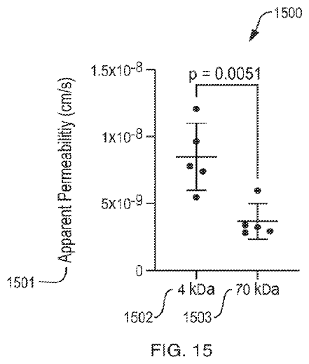

FIG. 15 shows a plot 1500 representing the apparent permeability 1501 of a dextran size of 4 kDa 1502 and 70 1503. The calculated apparent permeabilities (in cm/s) show a significant difference across dextran sizes. Data represents apparent permeability values calculated from fluorophore concentration values sensed by noninvasive fiber photometry on-chip, showing that fluorophore diffusion may be tracked noninvasively on-chip.

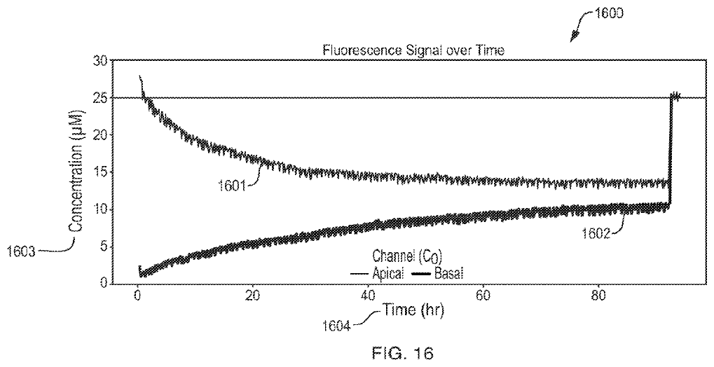

FIG. 16 shows a plot 1600 representing the non-invasive sensing of fluorescein dextran concentration on-chip. The plot 1600 shows the fluorophore concentration 1603 over time 1604 by comparing measured fluorescence intensities to intensity values at known concentrations. This plot 1600 sensed the fluorophore concentration from the apical 1601 and basal 1602 organ-chip channels over a period of four days. The diffusion of 70 kDa fluorescein dextran from the apical channel to the basal channel was observed as a respective decrease, and consequential increase, in sensed fluorophore concentration. These measurements were recorded under static conditions.

FIG. 17 shows a plot 1700 representing noninvasive sensing of lucifer yellow across three OoC simultaneously using high-density fiber optic cables. The diffusion of lucifer yellow from the apical 1701a-c channel to the basal 1702 a-c channel was observed as an increase in sensed fluorescence intensity in the basal channel. These measurements were recorded under flow conditions where apical and basal channels were perfused at a rate of 3 uL/min.

### REFERENCES

[1] Cannon, J., & Greenamyre, T. (2010) Neurotoxic in-vivo models of Parkinson's disease recent advances. Progress in Brain Research 184:17-33.
[2] Young, E. (2013) Cells, tissues, and organs on-chips: challenges and opportunities for the cancer tumor microenvironment. Integr. Biol. 5: 1096-1109.
[3] Horvath, P., et al. (2016) Screening out irrelevant cell-based models of disease. Nature Reviews Drug Discovery 15: 751-769.
[4] Ching, T., et al. (2021) Bridging the academia-to-industry gap: organ-on-a-chip platforms for safety and toxicology assessment. Trends in Pharmacological Sciences 42: 715-728.
[5] Zhang, B., & Radisic, M. (2017) Organ-on-a-chip devices advance to market. Lab Chip 17: 2395-2420.
[6] Wheeler, A. (2019) Directive to Prioritize Efforts to Reduce Animal Testing. United States Environmental Protection Agency.
[7] Andringa, J., et al. (2016) Transition to non-animal research. Netherlands National Committee for the protection of animals used for scientific purposes.
[8] Zhang, B., et al. (2018) Advances in organ-on-a-chip engineering. Nature Reviews Materials 3: 257-278.
[9] Vunjak-Novakovic, G., et al. (2021) Organs-on-a-chip models for biological research. Cell 18: 4597-4611.
[10] Kim, H., et al. (2012) Human gut-on-a-chip inhabited by microbial flora that experiences intestinal peristalsis-like motions and flow. Lab Chip 12: 2165-2174.
[11] Bhise, N., et al. (2016) A liver-on-a-chip platform with bioprinted hepatic spheroids. Biofabrication 8: 014101.
[12] Grant, J., et al. (2022) Establishment of physiologically relevant oxygen gradients in microfluidic organ chips. Lab Chip 22: 1584-1593.
[13] Prot, J., et al. (2011) Improvement of HepG2/C3a cell functions in a microfluidic biochip. Biotechnology and Bioengineering 7: 1704-1715.
[14] Marrero, D., et al. (2021) Gut-on-a-chip: mimicking and monitoring the human intestine. Biosensors and Bioelectronics 181: 113156.
[15] Caballero, D., Reis, R., & Kundu, S. (2022) Boosting the clinical translation of organ-on-a-chip technology. Bioengineering 9: 549.
[16] Kieninger, J., et al. (2018) Microsensor systems for cell metabolism-from 2D culture to organon-chip. Lab Chip 9: 1274-1291.
[17] Dornhof, J., et al. (2021) Microfluidic organ-on-chip system for multi-analyte monitoring of metabolites in 3D cell cultures. Lab Chip 22: 225-239.
[18] Wong, J., et al. (2020) Integrated electrochemical measurement of endothelial permeability in a 3D hydrogel-based microfluidic vascular model. Biosensors and Bioelectronics 147: 11757.
[19] Zhang, Y., et al. (2017) Multisensor-integrated organs-on-chips platform for automated and continual in situ monitoring of organoid behavior. PNAS 114: 2293-2302.
[20] Weltin, A., et al. (2014) Cell culture monitoring for drug screening and cancer research: a transparent, microfluidic, multi-sensor microsystem. Lab Chip 14: 138-146.
[21] Kim, C., et al. (2016) Simultaneous fast measurement of circuit dynamics at multiple sites across the mammalian brain. Nature Methods 13: 325-332.
[22] Sych, Y., et al. (2019) High-density multi-fiber photometry for studying large-scale brain circuit dynamics. Nature Methods 16: 553-660.
[23] Martianova, E., Aronson, S., & Proulx, C. (2019) Multi-fiber photometry to record neural activity in freely-moving animals. J. Vis. Exp. 152: e60278.
[24] Muir, J., et al. (2018) In vivo fiber photometry reveals signature of future stress susceptibility in nucleus accumbens. Neuropsychopharmacology 43: 255-263.
[25] Formozov, A., Dieter, A., & Wiegert, J. (2023) A flexible and versatile system for multi-color fiber photometry and optogenetic manipulation. Cell Reports Methods 3: 100418.
[26] Hosic, S., et al. (2021) Rapid prototyping of multilayer microphysiological systems. ACS Biomater. Sci. Eng. 7: 2949-2963.
[27] Zidan, H., & Abu-Elnader, M. (2004) Structural and optical properties of pure PMMA and metal chloride-doped PMMA films. Physica B 355: 308-317.
[28] Zhao, W., et al. (2019) Lucifer Yellow-A Robust Paracellular Permeability Marker in a Cell Model of the Human Blood-brain Barrier. J. Vis. Exp. 150: e58900.
[29] Himanshu, R., et al. (2013) The impact of permeability enhancers on assessment for monolayer of colon adenocarcinoma cell line (Caco-2) used in in vitro permeability assay. J. Drug Delivery & Therapeutics 3: 20-29.
[30] Preininger, C., Klimant, I., & Wolfbeis, O. (1994) Optical fiber sensor for biological oxygen demand. Anal. Chem. 66: 1841-1846.
[31] Zirath, H., et al. (2021) Bridging the academic-industrial gap: application of an oxygen and pH sensor-integrated lab-on-a-chip in nanotoxicology. Lab Chip 21: 4237-4248.
[32] Steinegger, A., Wolfbeis, O., & Borisov, S. (2020) Optical sensing and imaging of pH values: spectroscopies, materials, and applications. Chem. Rev. 120: 12357-12489.
[33] Hsu, H., et al. (2018) A Method for Determination and Simulation of Permeability and Diffusion in a 3D Tissue Model in a Membrane Insert System for Multi-well Plates. J. Vis. Exp. 132: 56412.
[34] Hubatsch, I., Ragnarsson, E., & Artursson, P. (2007) Determination of drug permeability and prediction of drug absorption in Caco-2 monolayers. Nature Protocols 2: 2111-2119.
[35] Eichele, D., & Kharbanda, K. (2017) Dextran sodium sulfate colitis murine model: An indispensable tool for advancing our understanding of inflammatory bowel diseases pathogenesis. World J. Gastroenterol. 23: 6016-6029.
[36] Wong, J., et al. (2020) Integrated electrochemical measurement of endothelial permeability in a 3D hydrogel-based microfluidic vascular model. Biosensors and Bioelectronics 147: 111757.
[37] Brown, J., et al. (2016) Metabolic consequences of inflammatory disruption of the blood-brain barrier in an organ-on-chip model of the human neurovascular unit. Journal of Neuroinflammation 13: 306-323.
[38] Rienecker, K., Poston, R., & Saha, R. (2020) Merits and limitations of studying neuronal depolarization dependent processes using elevated external potassium. ASN Neuro. 12: 1-17.
[39] Cameron, M., et al. (2016) Calcium imaging of AM dyes following prolonged incubation in acute neuronal tissue. PLOS ONE 11: e0155468.

The teachings of all patents, published applications, and references cited herein or in the poster being filed herewith are incorporated by reference in their entirety.

While example embodiments have been particularly shown and described, it will be understood by those skilled in the art that various changes in form and details may be made therein without departing from the scope of the embodiments encompassed or contemplated herein or in the poster being filed herewith.

What is claimed is:

1. A fluidic device, comprising:
a first layer having a first interface side and an opposing side, the first interface side defining a first complementary portion of a cavity;
a second layer having a second interface side and an opposing side, the second interface side defining a second complementary portion of the cavity;
the first and second layers configured to be coupled together via magnetic attraction, said coupled layers mating the first and second interface sides together; and
the mating of the first and second interface sides forming a cavity, at least a portion of which is defined by the complementary portions, the first and second complementary portions in a coupled arrangement configured to contain a fluid.

2. The fluidic device of claim 1, further comprising one or more gaskets affixed to the interface sides and arranged to form a perimeter around the first complementary portion of the cavity and the second complimentary portion of the cavity.

3. The fluidic device of claim 1, further comprising a third layer positioned between the first layer and second layer, the positioned third layer creating an upper channel in either the first layer or second layer, or a lower channel in either the first layer or second layer.

4. The fluidic device of claim 1, further comprising:
internal microchannels defined at least in part by the cavity formed by the mating of the first and second interface sides;
at least one valve operably coupled to either the first or the second layer, and operable from the opposing side of either the first or the second layer; and
wherein the at least one valve is configured to redirect or block fluid flow between the internal microchannels.

5. The fluidic device of claim 1, wherein:
four edges each located perpendicular to the opposing side of the first layer and the opposing side of the second layer such that a rectangular prism is formed by the mating of the first interface side and the second interface side; and
at least one interlocking joint positioned on an edge, the at least one interlocking joint configured to allow the fluidic device to be reconfigurably interlocked with an interlocking joint of another device.

6. The fluidic device of claim 1, further comprising:
a permanent magnet or an electromagnet coupled to, or captive in, the first layer and producing a magnetic attraction in combination with a metallic component coupled to the second layer, or in combination with a permanent magnet or electromagnet coupled to, or captive in, the second layer.

7. The fluidic device of claim 6, wherein the magnetic attraction has a flux density, and wherein flux density has a strength adjustably variable by a user.

8. A fluidic device comprising:
a structure defining at least one internal cavity configured to contain a fluid;
at least one port defined by the structure and oriented in optical arrangement with the at least one internal cavity, the at least one port oriented in a direction enabling optical viewing into the cavity;
the at least one port configured to couple an optical transmission channel to the fluidic device in a fixed position relative to the at least one cavity, the optical transmission channel configured to transmit optical radiation to or receive optical radiation from the fluid in the at least one cavity.

9. The fluidic device of claim 8, further comprising:
at least one membrane layer internal to the structure that is positioned to create a plurality of internal cavities in the structure, each of the plurality of internal cavities having respective ports positioned in line with at least one respective cavity.

10. The fluidic device of claim 8, wherein the fluid contains photoluminescent materials that are capable of being excited by light.

11. The fluidic device of claim 8, further comprising:
at least one optical sensing device operably coupled to the at least one port configured to record excitations from the photoluminescent materials in the fluid.

12. The fluidic device of claim 8, wherein the structure, and at least one internal cavity defined therein, is configurable to operate as an Organ on Chip.

13. The fluidic device of claim 8, wherein the optical radiation transmitted to or received from the structure is automatically tuned based on a benchmark sample.

14. The fluidic device of claim 8, further comprising:
the optical transmission channel; and
a single-mode wavelength filter element disposed within the optical transmission channel.

15. The fluidic device of claim 8, further comprising:
the optical transmission channel; and
a multi-mode wavelength filter element disposed within the optical transmission channel.

16. The fluidic device of claim 8, further comprising:
a plurality of ports positioned in optical arrangement with at least one of the at least one internal cavity; and wherein
each port of the plurality of ports is configured to receive a set of optical fibers arranged in a direction of a flow path defined by the at least one cavity, the optical fibers configured to record longitudinal changes of fluid flow over time in the flow path.

17. The fluidic device of claim 8, further comprising gaskets positioned at the at least one port to seal the port fluidically.

18. The fluidic device of claim 8, further comprising:
a processor operably coupled to the fluidic device and configured to process optical data collected as a function of a signal captured via the optical transmission channel.
